(19) United States
(12) Patent Application Publication
(10) Pub. No.: US 2026/0232379 A1
(43) Pub. Date: Aug. 13, 2026

(54) SYSTEMS AND METHODS FOR INVASIVE DIAGNOSTIC PLANNING

(71) Applicant: KONINKLIJKE PHILIPS N.V., EINDHOVEN (NL)

(72) Inventors: ARJEN VAN DER HORST, TILBURG (NL); MICHAEL GRASS, BUCHHOLZ IN DER NORDHEIDE (DE); HANS CHRISTIAN HAASE, HAMBURG (DE); HANNES NICKISCH, HAMBURG (DE); HOLGER SCHMITT, LUETJENSEE (DE); MANINDRANATH VEMBAR, TWINSBURG, OH (US)

(21) Appl. No.: 19/155,430

(22) PCT Filed: Feb. 6, 2024
(86) PCT No.: PCT/EP2024/052949

§ 371 Date: Aug. 11, 2025

Related U.S. Application Data
(60) Provisional application No. 63/445,024, filed on Feb. 13, 2023.

(51) Int. Cl. A61B 34/10; G06T 7/00; G06V 10/764; G06V 10/82; G16H 20/40; G16H 30/20

(52) U.S. Cl. CPC A61B 34/10; G06T 7/0014; G06V 10/764; G16H 20/40; G16H 30/20; A61B 2034/105; G06T 2207/10081; G06T 2207/20044; G06T 2207/20081; G06T 2207/20084; G06T 2207/30104; G06T 2207/30172; G06V 10/82; G06V 2201/03

(57) ABSTRACT

A method is provided for implementing invasive diagnostic testing in advance of a procedure. Such a method includes retrieving an image including at least a portion of a blood vessel, and extracting cross-sectional profiles at distinct locations along the blood vessel from the image. The method then proceeds to generate a hemodynamic model based on the image and at least partially based on the cross-sectional profiles. The hemodynamic model models blood flow at each of the distinct locations for which cross-sectional profiles have been extracted. The method then extracts, from the image, a determination of plaque features and generates or retrieves an initial treatment plan based at least partially on the hemodynamic model and the determined plaque features. The method then determines, based on the hemodynamic model and the determination of plaque features, that a defined invasive diagnostic test is likely to change the initial treatment plan.

### FIELD OF THE INVENTION

The present disclosure generally relates to systems and methods for invasive diagnostic planning. In particular, the present disclosure relates to the use of CT-guided modeling to determine whether the use of an invasive diagnostic device is likely to add value and to support invasive diagnostic device selection for percutaneous coronary interventions (PCI).

### BACKGROUND

Coronary interventions, such as percutaneous coronary interventions (PCI), are often planned based on imaging. Such imaging, which may be cardiac CT imaging, is often used as a first-line test for coronary artery disease (CAD), and more and more patients with prior CT exams are scheduled regularly for diagnostic and interventional cathlab procedures.

Spectral CT and spectral CT angiography allow for better coronary assessment due to the material separation between contrast agent and calcified lesions. Further, such spectral CT imaging allows for better robustness to imaging artifacts, such as calcium blooming. Based on the improved material separation, high-risk plaque characteristics, such as positive remodeling, low attenuation plaque, spotty calcification, and napkin rink signs are visible.

However, in order to plan procedures, medical teams typically gather all diagnostic information initially and then create a surgical plan. Accordingly, modern procedures result in determining whether to perform invasive diagnostic functional and imaging measurements without first determining if such invasive procedures are necessary or likely to add value. Such procedures may include intravascular ultrasound (IVUS), optical coherence tomography (OCT), an angiography-based pressure or flow test, or a guide-wire based pressure or flow test.

As such, there is a need for a system and pre-operative method for utilizing imaging, such as CT imaging, in order to determine and plan an appropriate coronary intervention. There is a further need for such a system and method that can determine whether the CT imaging is sufficient to finalize such a plan, or if additional invasive diagnostic functional and imaging measurements, such as IVUS, OCT, pressure/flow, and angiography-based pressure or flow tests are required to improve such planning.

### SUMMARY

Methods and systems are provided for planning coronary interventions, such as percutaneous coronary interventions (PCI). Modern interventions are often planned based on spectral CT imaging that is increasingly used as a first-line test for CAD. However, diagnoses are often confirmed with, and interventions are often planned based on, additional invasive procedures.

To make best use of available resources, and to better manage risk to the patient, it is beneficial to determine whether treatment planning can be based on the CT data alone, or if additional invasive diagnostic functional and imaging measurements are required. Such more invasive procedures may include intravascular ultrasound (IVUS), optical coherence tomography (OCT), an angiography-based pressure or flow test, or a guide-wire based pressure or flow test.

Because there are benefits to avoiding unnecessary invasive procedures, the system and method described herein provide guidance as to whether a treatment plan can be made based on a CT image or CT angiography (CTA), or if further invasive assessment would be valuable. This guidance may be based on a determination as to how likely the additional invasive assessment is to alter a treatment plan proposed.

Many embodiments rely on spectral CT imaging, since such spectral CT imaging has the advantage that it improves local image contrast by varying the image reconstruction properties across the image depending on the underlying tissue, such that the visual assessment of the coronary artery morphology becomes more accurate. Such spectral CT imaging can then be used to model blood flow in the coronary artery, which can, in turn, be used to evaluate whether further information from more invasive diagnostic procedures are likely to alter a treatment plan.

In some embodiments, a method is provided for implementing invasive diagnostic testing in advance of a procedure. Such a method comprises retrieving at least one image, the image including at least a portion of a blood vessel, typically a coronary artery, and extracting from the at least one image, a plurality of cross-sectional profiles at distinct locations along the blood vessel.

The method then proceeds to generate a hemodynamic model based on the at least one image and at least partially based on the cross-sectional profiles. Such a hemodynamic model models blood flow at each of the distinct locations for which cross-sectional profiles have been extracted.

The method then extracts, from the at least one image, a determination of plaque features and generates or retrieves an initial treatment plan based at least partially on the hemodynamic model and the determined plaque features.

The method then determines, based on the hemodynamic model and the determination of plaque features, that a defined invasive diagnostic test is likely to change the initial treatment plan.

In some embodiments, the determination that the defined invasive diagnostic test is likely to change the initial treatment plan is by an AI-based algorithm trained on historic patient case data and corresponding risk profile maps.

In some embodiments, the method includes generating a recommendation that the defined invasive diagnostic test be performed.

In some embodiments, the defined invasive diagnostic test is one of a plurality of potential invasive diagnostic tests. The defined invasive diagnostic test is then the test of the plurality of potential invasive diagnostic tests determined to be most likely to change a diagnosis or alter a surgical plan for the procedure.

In some such embodiments, the plurality of potential invasive diagnostic tests includes at least one of an intravascular ultrasound (IVUS), optical coherence tomography (OCT), an angiography-based pressure or flow test, and a guide-wire based pressure or flow test.

In some embodiments, the determination that the defined invasive diagnostic test is likely to change the initial treatment plan is by modeling the performance of the defined invasive diagnostic test in the hemodynamic model.

In some embodiments, the at least one image is a CT image, and the cross-sectional profiles extracted from the image are perpendicular to local centerlines extracted from the corresponding CT image. In some such embodiments, the CT image is a contrast-based image, and the method further includes generating a binary image mask from the contrast-based image, skeletonizing the corresponding CT image, and deriving at least one localized centerline from the skeletonized image.

In some embodiments, the hemodynamic model determines blood pressure, blood flow, and shear stress at locations corresponding to each cross-sectional profile.

In some embodiments, the CT image is a spectral CT image, and the determination of plaque features includes determining a type of plaque and a corresponding morphological pattern at each cross-sectional profile.

In some such embodiments, a depth of calcification at each cross-sectional profile is determined based on a distance of the calcification from the centerline. The centerline then corresponds to a center of a corresponding lumen at the cross-sectional profile.

In some embodiments the method proceeds to display, to a user, a map of plaque at each cross-sectional profile or a map of high-risk features in the hemodynamic model.

In some embodiments, the method determines, from the plaque features, a plaque rupture risk. In such embodiments, the initial treatment plan is based at least partially on the plaque rupture risk.

In some embodiments, the method determines that a defined invasive diagnostic test is likely to change the initial treatment plan by determining a percentage likelihood that the defined invasive test will change the initial treatment plan and comparing the determined likelihood to a threshold percentage likelihood.

In some embodiments, the method includes modeling an implementation of potential invasive diagnostic tests in the context of the hemodynamic model. For each potential invasive diagnostic test, the method then generates a likelihood that the corresponding diagnostic test will change the initial treatment plan. The defined invasive diagnostic test is then one of the plurality of potential invasive diagnostic tests.

In some such embodiments, the method further includes displaying, to a user, the likelihood associated with each of the potential diagnostic tests.

In some embodiments, generating the likelihood is by an AI-based algorithm trained on historic patient case data and corresponding risk profile maps.

### BRIEF DESCRIPTION OF THE DRAWINGS

FIG. 1 is a schematic diagram of a system according to one embodiment of the present disclosure.

FIG. 2 illustrates a method for invasive diagnostic testing in accordance with this disclosure.

### DETAILED DESCRIPTION OF THE PREFERRED EMBODIMENTS

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

This disclosure describes the best mode or modes of practicing the invention as presently contemplated. This description is not intended to be understood in a limiting sense, but provides an example of the invention presented solely for illustrative purposes by reference to the accompanying drawings to advise one of ordinary skill in the art of the advantages and construction of the invention. In the various views of the drawings, like reference characters designate like or similar parts.

It is important to note that the embodiments disclosed are only examples of the many advantageous uses of the innovative teachings herein. In general, statements made in the specification of the present application do not necessarily limit any of the various claimed disclosures. Moreover, some statements may apply to some inventive features but not to others. In general, unless otherwise indicated, singular elements may be in plural and vice versa with no loss of generality.

A pre-operative method is described for planning a surgical procedure based on CT imaging. The method involves extracting cross-sectional profiles at distinct locations along the coronary artery and generating a hemodynamic model based on the CT imaging. Such a hemodynamic model models blood flow at the distinct locations for which cross-sectional profiles have been extracted.

Typically, if a patient is scheduled for a coronary intervention, such as a percutaneous coronary intervention (PCI), the method described herein may be applied to plan the underlying procedure or intervention. In doing so, the hemodynamic model may be used to determine whether the information derived from the CT imaging is sufficient for planning the intervention, or if additional information would be valuable. Such a determination may be based on determining how likely additional information obtainable by way of invasive diagnostic testing is to alter a diagnosis or surgical plan for the procedure.

In some embodiments, pre-intervention imaging may take a form other than CT, such as the spectral CT imaging discussed above. In medical imaging other than CT, such as Magnetic Resonance Imaging (MRI) or Positron Emission Tomography (PET), different methods may be used for processing images, and resulting images or image data may take different forms. In this disclosure, embodiments are discussed in terms of CT imaging. However, it will be understood that the methods and systems described herein may be used in the context of other imaging modalities as well.

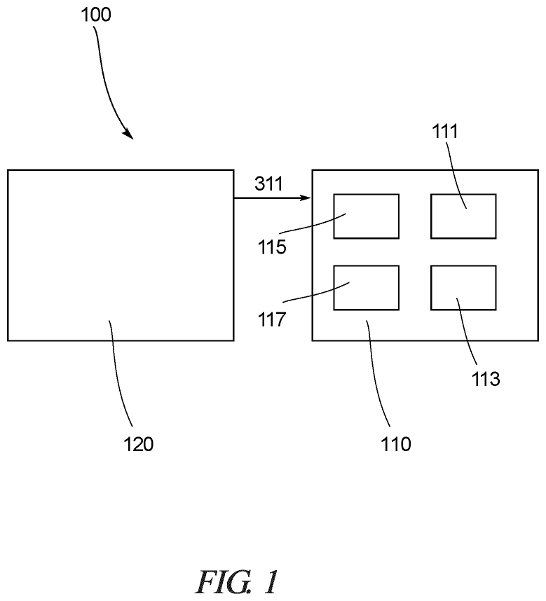

FIG. 1 is a schematic diagram of a system 100 according to one embodiment of the present disclosure. As shown, the system 100 typically includes a processing device 110 and an imaging device 120.

The processing device 110 may apply processing routines to images or measured data, such as projection data, received from the imaging device 120. The processing device 110 may include a memory 113 and processor circuitry 111. The memory 113 may store a plurality of instructions. The processor circuitry 111 may couple to the memory 113 and may be configured to execute the instructions. The instructions stored in the memory 113 may comprise processing routines, as well as data associated with processing routines, such as machine learning algorithms, and various filters for processing images. While all data is described as being stored in the memory 113, it will be understood that in some embodiments, some data may be stored in a database, which may itself either be stored in the memory or stored in a discrete system.

The processing device 110 may further include an input 115 and an output 117. The input 115 may receive information, such as images or measured data, from the imaging device 120. The output 117 may output information, such as processed images, to a user or a user interface device. The output 117 similarly may output determinations generated by the method described below, such as recommendations and risk determinations, as well as likelihoods that specified tests are likely to change a diagnosis or treatment. The output may include a monitor or display which may display additional information or a model updated in real-time.

In some embodiments, the processing device 110 may relate to the imaging device 120 directly. In alternate embodiments, the processing device 110 may be distinct from the imaging device 120, such that it receives images or measured data for processing by way of a network 311 or other interface at the input 115.

In some embodiments, the imaging device 120 may include an image data processing device, and a spectral or conventional CT scanning unit for generating the CT projection data when scanning an object (e.g., a patient). Further, the imaging device 120 may be set up for coronary CT angiography. As such, the imaging may be performed with contrast, and the image timing may be set up in order to track fluid flow in blood vessels.

In addition to conventional and spectral CT images, the method may rely on multiple spectral image results, photon counting CT images, or dark field CT images.

While a system is shown including an imaging device 120 and a processing device 110, it will be understood that the method may be implemented directly on a processing device, as in the context of an image received by way of a network 311 at the input 115. The methods described herein involve processing an image as a component of evaluating and planning potential interventions, generally in the context of a procedure, such as stenting. As noted above, typically, prior to such a procedure, imaging is performed. As such, previously generated imaging may be retrieved by way of the input 115 and evaluated prior to or in place of obtaining a new image.

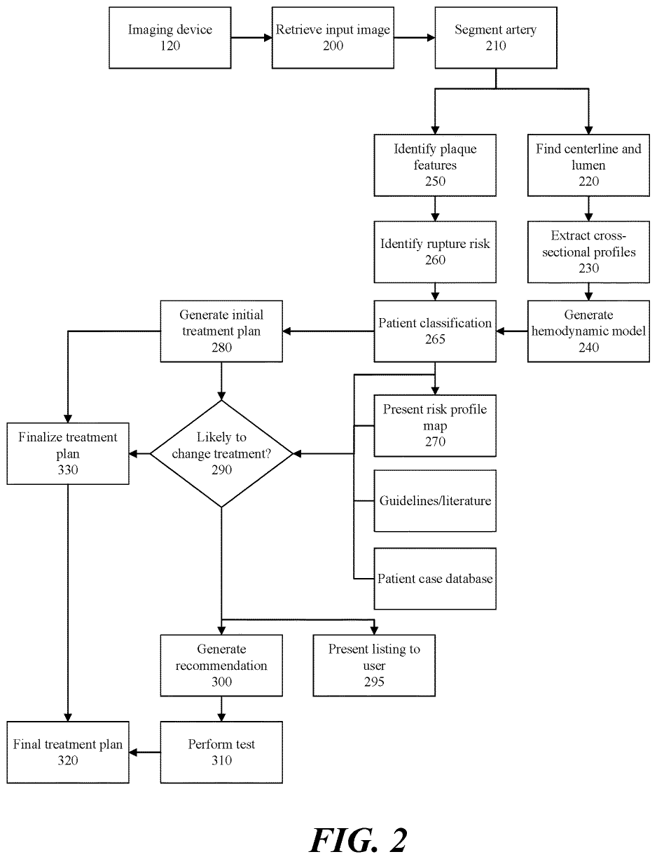

FIG. 2 illustrates a method for implementing invasive diagnostic testing in accordance with this disclosure. As shown, in implementing the method, the system 100 described may first retrieve (200), at an input 115, at least one image from an imaging device 120. The image includes at least a portion of a coronary artery of the patient. The retrieved image is typically by CT imaging, such as a traditional CT scan or non-invasive coronary CT angiography, which may be used to evaluate plaque in the coronary artery. Such CT imaging may be spectral CT imaging, which provides additional information, as discussed above. When referring to an image, it will be understood that more than one image may be relied on, and that a CT image may be a three-dimensional image built from a large number of scans.

Once such imaging is retrieved (at 200), the method proceeds to segment the coronary artery (210) in order to support an analysis. This may include finding a centerline and lumen contours for coronary arteries (220). This may be done, for example, using spectral reconstructions using established intensity thresholds in mono-energetic CT images, or with the use of iodine-based images to create binary image masks. Skeletonization may then be applied to derive centerlines, such that after skeletonization of the corresponding CT image, at least one localized centerline may be extracted therefrom.

At each centerline location, a hypothetical healthy vessel diameter can be estimated from the closest non-diseased proximal and distal locations. The method may then create or extract (230) from the image a plurality of cross-sectional profiles, or patches, corresponding to distinct locations along the coronary artery. Such cross-sectional profiles are typically perpendicular to the local centerline direction (found at 220) previously extracted from the corresponding CT image. In some embodiments, photon-counting CT may be used for this procedure due to superior accuracy.

Once cross-sectional profiles have been created or extracted (at 230), the method proceeds to generate a hemodynamic model (240). Such a model is based on the image itself along with the segmentation (at 210) and the derived cross-sectional profiles (at 230). The hemodynamic model models blood flow at each of the distinct locations for which cross-sectional profiles have been created or extracted. In some embodiments, in addition to the underlying blood flow, the hemodynamic model models blood pressure and shear stress at locations corresponding to each cross-sectional profile (extracted at 230).

Separately, the method returns to the segmentation (at 210) and extracts from the at least one image (retrieved at 200) a determination of plaque features in segments identified by the segmentation (250). In some embodiments, the plaque features may be identified and processed at locations corresponding to each of the cross-sectional profiles derived (at 230).

The identified plaque features (250) may include an identification of a longitudinal position of the plaque in the artery, as well as location and classification of the plaque. For example, the plaque may be classified as soft, mixed, or calcified. Such a determination may be based on spectral CT image intensities, where a spectral CT image is used. Further, such determination may be based on known typical morphological patterns for plaque, such as a napkin ring sign.

In some embodiments, a depth of calcification of plaque may similarly be estimated by considering the plaque features (250) in the context of the centerline and lumen contours for the coronary arteries (identified at 220). Accordingly, a determination whether calcification is in the intimal layer or medial layer, for example, can be estimated from the distance of the calcification from the center of the lumen. Accordingly, the determination of the plaque features (at 250) may be a combination of determining the type of plaque and a corresponding morphological pattern at each cross-sectional profile.

In some embodiments, calcium content quantification may be in terms of CAN=Non-Calcified/CAL=Low or mixed level of Calcification/CAH=Highly Calcified.

High-Risk Plaque (HRP) features may be identified as well. Such high risk features may include LA=Low Attenuation plaque <30HU/NR=Napkin-Ring-sign i.e. (low attenuation core surrounded by rim-like higher attenuation and <130HU)/PR=Positive Remodeling (remodeling idx>1.1=10% increase in CSA)/SC=Spotty Calcification (calcified foci between 1-3 mm and >130HU) Other patterns may be identifiable as well. For example, plaque may be classified as DC=Dense Calcification (massively calcified, other plaque components negligible)/FM=Fibro-calcified mix (mixture of calcified foci and fibrous components)/FP=Fibrous Plaque (exclusively fibrous, other plaque components negligible) Other features of interest may include Thrombus/Peri-coronary inflammation (e.g., via pFAI).

Once the plaque features are identified (at 250), such plaque features may be used to identify a rupture risk (260), which may in turn be used to guide treatment. Once the plaque features are fully evaluated (at 250, 260) and the cross-sectional profiles are extracted (at 230) and utilized in the context of the hemodynamic model (at 240), the results may be combined to generate a patient classification (265) for a patient.

In some embodiments, these findings may be presented independently to a user (at 270) on a cross sectional basis, for example, using color coding on curved or multiplanar reformats of the vessel. The presentation to the user may be in the context of a risk profile map or other presentation of high-risk features. Accordingly, the method may display a map of plaque at each cross-sectional profile or a map of high-risk features in the hemodynamic model.

Once a hemodynamic model is generated (at 240) and plaque features have been identified (at 250), an initial treatment plan is generated or retrieved (280) based at least partially on the hemodynamic model and the plaque features. Such a treatment plan may be generated manually and thereby provided by a clinician utilizing the method described, or it may be automatically generated by the method. In some embodiments, the treatment plan may be independently generated by a clinician and provided regardless of whether the clinician generating the treatment plan is utilizing the method.

Once an initial treatment plan is generated or retrieved (at 280), the method proceeds to determine (290) based on the hemodynamic model and the determination of plaque features, whether one or more invasive diagnostic test is likely to change the initial treatment plan already generated or retrieved (at 280). If an invasive test is unlikely to provide additional information that will change a course of action proposed by a clinician or recommended by the method, the value of such a test may be minimal. Accordingly, even if such a test is likely to provide additional information, if such information is not likely to change a treatment plan, or in some cases, a diagnosis, such a test should be avoided. In this way, the method may determine whether an invasive diagnostic test provides sufficient value to justify the cost, time, effort, and/or risk associated with such a test.

As part of the determination (at 290) of whether one or more invasive diagnostic test is likely to change the initial treatment plan, the method may independently evaluate each of a plurality of potential invasive diagnostic tests. Each such test may be, for example, an intravascular ultrasound (IVUS), optical coherence tomography (OCT), an angiography-based pressure or flow test, or a guide-wire based pressure or flow test. In some embodiments, the determination (at 290) may include an evaluation for each such test whether such a test is likely to change the initial treatment plan. In some embodiments, the method may further evaluate tests of a single type performed with different testing parameters.

Once each potential invasive diagnostic test is evaluated, each such test may be assigned a likelihood that the diagnosis and treatment plan, such as the initial treatment plan, will be altered if the corresponding diagnostic test is performed as a follow-up evaluation.

In some embodiments, a listing of potential invasive diagnostic tests along with corresponding likelihoods that such a test will change the diagnosis and treatment plan may be presented to the user (295). In other embodiments, the method may instead select a defined invasive diagnostic test selected from the plurality of potential tests, where the defined test is that test most likely to change the initial treatment plan. Such likelihoods may be defined in terms of a percentage likelihood that the corresponding invasive test will change the initial treatment plan, or may be presented in some other way. In such embodiments, a threshold value for a metric measuring likelihood may be provided. For example, a threshold percentage likelihood may be provided. As such, the likelihood assigned to a particular test may be compared to the threshold to determine if the corresponding test, or any of the plurality of potential invasive diagnostic tests, are to be recommended.

In some embodiments, the determination (at 290) of whether the one or more invasive diagnostic test is likely to change the initial treatment plan is based on an AI-based algorithm, such as a convolutional neural network (CNN). Such a CNN may be trained based on historic patient case data and corresponding risk profile maps. In such embodiments, the determination may be based on a risk profile map generated for the patient (at 270). Such historic patient case data may further include documentation on the corresponding initial diagnosis and treatment plan, any further invasive diagnostic measurements acquired during treatment, and final treatment documentation.

In some embodiments, the AI-based algorithm may be provided with guidelines and other literature associated with patient care. Such guidelines may be utilized to determine if information that could be made available by the one or more invasive diagnostic test is likely to change the initial diagnosis or treatment plan.

It is understood that while the method described herein utilizes CNNs, other machine learning techniques may be used as well. For example, generic neural networks, such as random forests, shallow predictors (support vector machines, or SVMs and gaussian processes, or GPs) or more advanced architectures, such as recurrent neural networks (RNNs) and transformer models, among others, may be used as well.

The method may be used in CT systems and imaging workstations and PACS viewers dedicated to coronary analysis using CCTA scans.

In some embodiments, the determination (at 290) that the defined invasive diagnostic test is likely to change the initial treatment plan is by modeling the performance of the defined invasive diagnostic test in the context of the hemodynamic model. In some embodiments, where multiple potential invasive diagnostic tests are considered, each such test may be modeled in the context of the hemodynamic model.

In some embodiments, the method may generate a recommendation that an invasive diagnostic test be performed. In such an embodiment the determination (at 290) may be that a defined invasive diagnostic test is likely to change the initial treatment plan. As such, the method may proceed to recommend (300) the defined invasive diagnostic test. During practice, a clinician utilizing the method may then proceed to perform the recommended invasive test (310) and create a final treatment plan (320). Where the method determines that an invasive diagnostic test is unlikely to change the initial treatment plan (at 280), the initial treatment plan may be finalized (at 330) as the final treatment plan (at 320).

In some embodiments, the determination (at 290) is based on specified factors understood from the literature. For example, in a case where certain sections have 3 or more high-risk plaque characteristics as determined based on the plaque features (at 250), the method may not advise an invasive physiology measurement, and may instead advise medical therapy or direct stenting if CT based fractional flow reserve analysis (CT-FFR) is less than 0.8.

Alternatively, where a section has less than 3 high-risk plaque characteristics and CT-FFR close to the cut-off point, the method may advise an invasive FFR.

Where structures are further from the vessel lumen, the method may suggest IVUS instead of OCT. Where indications of collaterals are found, the method may indicate that an angio based FFR or instantaneous wave-free ration (iFR) should not be used, and instead recommend a wire-based measurement. Where tandem lesions are identified, the method may advise iFR instead of FFR.

While the method is described herein in terms of coronary artery, it is understood that similar methods may be used in the context of peripheral artery disease, for pulmonary arteries, as well as for carotids or cerebral vessels (e.g., circle of Willis).

The methods according to the present disclosure may be implemented on a computer as a computer implemented method, or in dedicated hardware, or in a combination of both. Executable code for a method according to the present disclosure may be stored on a computer program product. Examples of computer program products include memory devices, optical storage devices, integrated circuits, servers, online software, etc. Preferably, the computer program product may include non-transitory program code stored on a computer readable medium for performing a method according to the present disclosure when said program product is executed on a computer. In an embodiment, the computer program may include computer program code adapted to perform all the steps of a method according to the present disclosure when the computer program is run on a computer. The computer program may be embodied on a computer readable medium.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All examples and conditional language recited herein are intended for pedagogical purposes to aid the reader in understanding the principles of the disclosure and the concepts contributed by the inventor to furthering the art, and are to be construed as being without limitation to such specifically recited examples and conditions. Moreover, all statements herein reciting principles, aspects, and embodiments of the disclosure, as well as specific examples thereof, are intended to encompass both structural and functional equivalents thereof.

Additionally, it is intended that such equivalents include both currently known equivalents as well as equivalents developed in the future, i.e., any elements developed that perform the same function, regardless of structure.

1. A computer-implemented method for implementing invasive diagnostic testing in advance of a procedure, comprising:
retrieving at least one image, the image including at least a portion of a blood vessel;
extracting, from the at least one image, a plurality of cross-sectional profiles at distinct locations along the blood vessel;
generating a hemodynamic model based on the at least one image and at least partially based on the cross-sectional profiles, the hemodynamic model modeling blood flow at each of the distinct locations for which cross-sectional profiles have been extracted;
extracting, from the at least one image, a determination of plaque features;
generating or retrieving an initial treatment plan based at least partially on the hemodynamic model and the determined plaque features;
determining, based on the hemodynamic model and the determination of plaque features, that a defined invasive diagnostic test is likely to change the initial treatment plan.

2. The method of claim 1, wherein the determination that the defined invasive diagnostic test is likely to change the initial treatment plan is by an Al-based algorithm trained on historic patient case data and corresponding risk profile maps.

3. The method of claim 1, wherein the blood vessel is a coronary artery.

4. The method of claim 1, wherein the defined invasive diagnostic test is one of a plurality of potential invasive diagnostic tests, and wherein the defined invasive diagnostic test is the test of the plurality of potential invasive diagnostic tests determined to be most likely to change a diagnosis or alter a surgical plan for the procedure.

5. The method of claim 4, wherein the plurality of potential invasive diagnostic tests includes at least one of an intravascular ultrasound (IVUS), optical coherence tomography (OCT), an angiography-based pressure or flow test, and a guide-wire based pressure or flow test.

6. The method of claim 1, wherein the determination that the defined invasive diagnostic test is likely to change the initial treatment plan is by modeling the performance of the defined invasive diagnostic test in the hemodynamic model.

7. The method of claim 1, wherein the at least one image is a CT image, and wherein the cross-sectional profiles extracted from the at least one image are perpendicular to local centerlines extracted from the corresponding CT image.

8. The method of claim 7, wherein the CT image is a contrast-based image, the method further comprising generating a binary image mask from the contrast-based image, skeletonizing the corresponding CT image, and deriving at least one localized centerline from the skeletonized image.

9. The method of claim 7, wherein the hemodynamic model determines blood pressure, blood flow, and shear stress at locations corresponding to each cross-sectional profile.

10. The method of claim 7, wherein the CT image is a spectral CT image, and wherein the determination of plaque features comprises determining a type of plaque and a corresponding morphological pattern at each cross-sectional profile.

11. The method of claim 10, further comprising determining a depth of calcification at each cross-sectional profile based on a distance of the calcification from the centerline, where the centerline corresponds to a center of a corresponding lumen at the cross-sectional profile.

12. The method of claim 10, further comprising displaying a map of plaque at each cross-sectional profile or a map of high-risk features in the hemodynamic model.

13. The method of claim 1, further comprising determining, from the plaque features, a plaque rupture risk, wherein the initial treatment plan is based at least partially on the plaque rupture risk.

14. The method of claim 1, wherein the determining that a defined invasive diagnostic test is likely to change the initial treatment plan is by determining a percentage likelihood that the defined invasive test will change the initial treatment plan and comparing the determined likelihood to a threshold percentage likelihood.

15. The method of claim 1, further comprising modeling an implementation of a plurality of potential invasive diagnostic tests in the context of the hemodynamic model, and for each potential invasive diagnostic test, generating a likelihood that the corresponding diagnostic test will change the initial treatment plan, wherein the defined invasive diagnostic test is one of the plurality of potential invasive diagnostic tests.

16. The method of claim 15, further comprising displaying the likelihood associated with each of the potential diagnostic tests.

17. The method of claim 15, wherein the generating of the likelihood is by an Al-based algorithm trained on historic patient case data and corresponding risk profile maps.

18. A system for implementing invasive diagnostic testing in advance of a procedure, comprising:
a memory for storing a plurality of instructions;
processor circuitry that couples with the memory and is configured to execute the instructions to:
retrieve at least one image, the image including at least a portion of a blood vessel;
extract from the at least one image a plurality of cross-sectional profiles at distinct locations along the blood vessel;
generate a hemodynamic model based on the at least one image and at least partially based on the cross-sectional profiles, the hemodynamic model modeling blood flow at each of the distinct locations for which cross-sectional profiles have been extracted;
extract, from the at least one image, a determination of plaque features;
generate or retrieve an initial treatment plan based at least partially on the hemodynamic model and the determined plaque features; and
determine, based on the hemodynamic model and the determination of the plaque features that a defined invasive diagnostic test is likely to change the initial treatment plan.

19. The system of claim 18, wherein the blood vessel is a coronary artery, and wherein the determination that the defined invasive diagnostic test is likely to change the initial treatment plan is by an Al-based algorithm trained on historic patient case data and corresponding risk profile maps.

20. The system of claim 18, wherein the defined invasive diagnostic test is one of a plurality of potential invasive diagnostic tests, and wherein the determination that the defined invasive diagnostic test is likely to change the initial treatment plan is by modeling an implementation of each of the plurality of potential invasive diagnostic tests in the context of the hemodynamic model and, for each potential invasive diagnostic test, generating a likelihood that the corresponding diagnostic test will change the initial treatment plan.
